(19) United States
(12) Patent Application Publication
(10) Pub. No.: US 2007/0190060 A1
(43) Pub. Date: Aug. 16, 2007

(54) Passive targeting of cytotoxic agents

(75) Inventors: Erwin Raymond Arsene Boghaert, Monroe, NY (US); Kiran Khandke, Nanuet, NY (US); Nitin Krishnaji Damle, Upper Saddle River, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/592,859

(22) PCT Filed: Mar. 15, 2005
(86) PCT No.: PCT/US05/08505

§ 371 Date: Sep. 14, 2006

Related U.S. Application Data
(60) Provisional application No. 60/553,112, filed on Mar. 15, 2004.

(51) Int. Cl. A61K 39/395

(52) U.S. Cl. 424/155.1; 424/178.1

(57) ABSTRACT

The present invention provides methods of treating cancer cells comprising administering to a patient in need thereof a therapeutically effective amount of a non-specific antibody conjugated to a cytotoxin, wherein the cancer cells do not express an antigen to which the non-specific antibody binds. In one embodiment, the non-specific antibody is an anti-CD33 antibody (e.g., hp67.6), an anti-CD22 antibody (e.g., g5/44), or an anti-CD20 antibody (e.g., rituximab). In another embodiment, the non-specific antibody does not bind a human antigen. The cancer cells treated can be, e.g., gastric, colon, non-small cell lung (NSCLC), breast, epidermoid, or prostate carcinoma cells. In one embodiment, the cytotoxin is calicheamicin. Calicheamicin can be conjugated to the non-specific antibody using a 4-(4′-acetylphenoxy)butanoic acid (AcBut) or (3-Acetylphenyl)acetic acid (AcPAc) linker. In another embodiment, the antibody to the non-specific antigen conjugated to a cytotoxin is administered in combination with a bioactive agent, e.g., an anti-cancer agent.

### FIELD OF THE INVENTION

The present invention relates to passive targeting of cytotoxic agents conjugated to a non-specific antibody.

### BACKGROUND OF THE INVENTION

The use of cytotoxic chemotherapy has improved the survival of patients suffering from various types of cancers. Used against select neoplastic diseases such as, e.g., acute lymphocytic leukemia in young people and Hodgkin lymphomas, cocktails of cytotoxic drugs can induce complete cures. Unfortunately, chemotherapy, as currently applied, does not result in complete remissions in a majority of cancers. Multiple reasons can explain this relative lack of efficacy. Among these, the low therapeutic index of most chemotherapeutics is a likely target for pharmaceutical improvement. The low therapeutic index reflects the narrow margin between the efficacious and toxic dose of a drug, which may prevent the administration of sufficiently high doses necessary to eradicate a tumor and obtain a curative effect.

One strategy to circumvent this problem is the use of a so-called magic bullet. The magic bullet consists of a cytotoxic compound that is chemically linked to an antibody. Binding a cytotoxic anticancer drug to an antibody that recognizes a tumor-associated-antigen can improve the therapeutic index of the drug. This antibody should ideally recognize a tumor-associated antigen (TAA) that is exclusively expressed at the surface of tumor cells. This strategy allows the delivery of the cytotoxic agent to the tumor site while minimizing the exposure of normal tissues. The antibody can deliver the cytotoxic agent specifically to the tumor and thereby reduce systemic toxicity.

Drug conjugates developed for systemic pharmacotherapy are target-specific cytotoxic agents. The concept involves coupling a therapeutic agent to a carrier molecule with specificity for a defined target cell population. Antibodies with high affinity for antigens are a natural choice as targeting moieties. With the availability of high affinity monoclonal antibodies, the prospects of antibody-targeting therapeutics have become promising. Toxic substances that have been conjugated to monoclonal antibodies include toxins, low-molecular-weight cytotoxic drugs, biological response modifiers, and radionuclides. Antibody-toxin conjugates are frequently termed immunotoxins, whereas immunoconjugates consisting of antibodies and low-molecular-weight drugs such as methotrexate and adriamycin are called chemoimmunoconjugates. Immunomodulators contain biological response modifiers that are known to have regulatory functions, such as lymphokines, growth factors, and complement-activating cobra venom factor (CVF). Radioimmunoconjugates consist of radioactive isotopes, which may be used as therapeutics to kill cells by their radiation or used for imaging. Antibody-mediated specific delivery of cytotoxic drugs to tumor cells is expected to not only augment their anti-tumor efficacy, but also to prevent nontargeted uptake by normal tissues, thus increasing their therapeutic indices.

Immunoconjugates using a member of the potent family of antibacterial and antitumor agents, known collectively as the calicheamicins or the LL-E33288 complex, were developed for use in the treatment of cancers. The most potent of the calicheamicins is designated γ₁^{l}, which is herein referenced simply as gamma. These compounds contain a methyltrisulfide that can be reacted with appropriate thiols to form disulfides, at the same time introducing a functional group such as a hydrazide or other functional group that is useful in attaching a calicheamicin derivative to a carrier. The calicheamicins contain an enediyne warhead that is activated by reduction of the —S—S— bond causing breaks in double-stranded DNA.

MYLOTARG®), also referred to as CMA-676 or CMA, is the only commercially available drug that works according to this principle. MYLOTARG® (gemtuzumab ozogamicin) is currently approved for the treatment of acute myeloid leukemia in elderly patients. The drug consists of an antibody against CD33 that is bound to calicheamicin by means of an acid-hydrolyzable linker. The disulfide analog of the semi-synthetic N-acetyl gamma calicheamicin was used for conjugation (U.S. Pat. Nos. 5,606,040 and 5,770,710, which are incorporated herein in their entirety). This molecule, N-acetyl gamma calicheamicin dimethyl hydrazide, is hereafter abbreviated as CM.

The use of the targeted cytotoxins in developing therapies for a wide variety of cancers has been limited both by the availability of specific targeting agents (carriers), as well as the conjugation methodologies which result in the formation of protein aggregates when the amount of the calicheamicin derivative that is conjugated to the carrier (i.e., the drug loading) is increased. For example, although calicheamicin is a potent chemotherapeutic with a low therapeutic index, it requires targeting to tumor cells for its use in the clinic. Dependence of this targeting strategy on specific antigen expression by tumor cells (active targeting) narrows its application range. Consequently, there is a need to devise new and improved methods for administering cytotoxins, for example, calicheamicin, conjugated to antibodies.

### BRIEF DESCRIPTION OF THE DRAWINGS

FIG. 1 demonstrates growth-inhibition of CD33 negative (CD33⁻) epidermoid carcinoma xenografts by hp67.6-AcBut-CalichDMH as a graph of tumor volume (mm³) versus period of tumor growth (days); FIG. 1A shows calicheamicin conjugated with an acid labile AcBut linker to hp67.6, FIG. 1B shows calicheamicin conjugated with an acid stabile Amide linker to hp67.6, and FIG. 1C shows free calicheamicin as a control. The symbols represent the average tumor volumes of 10 (PBS treatment) or 5 animals (calicheamicin or conjugate treatments), while error bars indicate the standard deviation. All the groups of mice received a regimen of 1 dose per mouse, given 3 times intraperitoneally with an interval of 4 days (Q4D×3). The number between brackets in the figure legends indicates the amount of calicheamicin (μg/mouse) given in a single dose as free drug or in conjugated form.

FIG. 2 demonstrates distribution of ¹²⁵I-labeled hp67.6-AcBut-CalichDMH as a function of time in CD33⁻ tumor bearing mice; FIG. 2A shows tumor, FIG. 2B shows blood, FIG. 2C show liver, FIG. 2D shows brain, FIG. 2E shows skin, FIG. 2F shows spleen, FIG. 2G shows striated muscle, FIG. 2H shows lung, FIG. 21 shows kidney, FIG. 2J shows heart, and FIG. 2K shows intestine. The amounts of hp67.6-AcBut-CalichDMH in the various normal tissues and tumor (A431) xenografts are presented relative to the amount of conjugate in total blood (open circles, Y1 axis, % Blood) or to the amount injected (closed circles, Y2 axis, % ID/g). All data points reflect the mean of 5 samples, while error bars indicate the standard deviations.

FIG. 3 demonstrates inhibition of tumor growth by passive targeting of calicheamicin using hp67.6, g5/44 and rituximab as carriers; FIG. 3A shows hp67.6-AcBut-CalichDMH and rituximab-AcBut-CalichDMH against N87 xenografts; FIG. 3B shows g5/44-AcBut-CalichDMH and hp67.6-AcBut-CalichDMH against N87, and FIG. 3C shows g5/44-AcBut-CalichDMH and hp67.6-AcBut-CalichDMH against MDAMB435/5T4. All the groups of mice treated with conjugate received a regimen of 1 dose of 4 μg CalichDMH per mouse, given 3 times intraperitoneally with an interval of 4 days (Q4D×3). Each point represents the average of n tumor measurements (see legend), while error bars reflect the standard deviation.

FIG. 4 demonstrates tumor growth inhibition by calicheamicin conjugates of HSA, PEGylated Fc and PEGylated hp67.6. The influence of MOPC-21-AcPAc-CalichDMH (FIG. 4A) on growth of A431 xenografts was compared to that of HSA-AcPAc-CalichDMH (FIG. 4B). Each point represents the average tumor volume of 5 (conjugate treatments) or 10 (PBS) xenografts. All the groups of mice received a regimen of 1 dose per mouse, given 3 times intraperitoneally with an interval of 4 days (Q4D×3). The number between brackets in the figure legends indicates the amount of calicheamicin (μg/mouse) given in a single dose. In FIG. 4C, the inhibition of A431 xenograft growth by calicheamicin conjugates of PEGylatedFc fragments was compared to that of hp67.6-AcBut-CalichDMH. Each point represents the average tumor volume of 5 (conjugate treatments) or 10 (PBS) xenografts. The efficacy of calicheamicin conjugates of hp67.6 and the PEGylated form of the antibody (hp67.6PEGB) is also shown against N87 tumor xenografts (FIG. 4D). Each point represents the average of tumor volumes for groups of 10 mice treated with PBS or hp67.6PEGB-AcBut-CalichDMH and the average of 7 for the group of mice treated hp67.6-AcBut-CalichDMH. In FIGS. 4C and 4D, all the groups of mice treated with conjugate received a regimen of 1 dose of 4 μg CalichDMH per mouse, given 3 times intraperitoneally with an interval of 4 days (Q4D×3). Error bars in all panels reflect the standard deviation.

FIG. 5 demonstrates inhibition of tumor growth by passive targeting of calicheamicin correlates with sensitivity of the tumor cells to calicheamicin in vitro. The sensitivity of tumor cell lines (X-axes, FIGS. 5A and 5B) to calicheamicin is presented as ED₅₀-value of either CalichDMH (Y1 axis, FIG. 5A) or hp67.6-AcBut-CalichDMH (Y1 axis, FIG. 5B). The height of each bar reflects the median of at least 3 independent ED₅₀ determinations. The sensitivity of the tumor xenografts to hp67.6-AcBut-CalichDMH is expressed as T/Cₘᵢₙ (Y2 axes, FIGS. 5A and 5B). The T/Cₘᵢₙ values (black diamonds, dashed exponential regression curve) are either determinations obtained from a single experiment (A431/Le^{y}, PC3MM2, KB 8.5, HT29) or the median of multiple experiments (N87 [n=6], PC14PE6 [n=3], LOVO [n=3], L2987 [n=2], MDAMB435/5T4 [n=2], A431 [n=3], LNCaP [n=2]). All the T/Cₘᵢₙ-values for hp67.6-AcBut-CalichDMH were determined following treatment with a regimen of 1 dose of 4 μg CalichDMH per mouse, given 3 times intraperitoneally with an interval of 4 days (Q4D×3).

### SUMMARY OF THE INVENTION

The present invention provides a method of treating cancer cells comprising administering to a patient in need thereof a therapeutically effective amount of a non-specific antibody conjugated to a cytotoxin, wherein the cancer cells do not express an antigen to which the non-specific antibody binds. In one embodiment, the non-specific antibody is an anti-CD33 antibody (e.g., hp67.6) and the cancer cells do not express CD33, an anti-CD22 antibody (e.g., g5/44) and the cancer cells do not express CD22, an anti-CD20 antibody (e.g., rituximab) and the cancer cells do not express CD20. In another embodiment, the non-specific antibody does not bind a human antigen. The cancer cells treated can be, for example, gastric carcinoma, colon carcinoma, non-small cell lung carcinoma (NSCLC), breast carcinoma, epidermoid carcinoma, or prostate carcinoma cells.

In one embodiment of the present methods, the cytotoxin is calicheamicin. Calicheamicin can be conjugated to the non-specific antibody using a 4-(4′-acetylphenoxy)butanoic acid (AcBut) or (3-Acetylphenyl)acetic acid (AcPAc) linker.

In another embodiment, the antibody to the non-specific antigen conjugated to a cytotoxin is administered in combination with a bioactive agent, for example, an anti-cancer agent.

### DETAILED DESCRIPTION OF THE INVENTION

The present application relates to the ability of cytotoxin-antibody conjugates causing tumor regression in various human tumors. These tumors did not display detectable amounts of the antigen recognized by the antibody. Thus, this treatment is referred to as passive targeting. In passive targeting, a conjugate of a non-specific antibody and a cytotoxin accumulate in a human tumor in the absence of detectable amounts of targeting antigen. Therefore, passive targeting of calicheamicin, for example, by means of an antibody or immunoglobulin carrier is a potential strategy to safely administer a therapeutically effective amount of calicheamicin.

A passive targeting strategy may be based on the enhanced permeability and retention effect (EPR) of a tumor. While not intending to be limited to any particular method of action, this effect may allows accumulation of particles or water-soluble macromolecules in a tumor because of the leakiness of the fenestrated endothelium of its blood vessels combined with an inadequate lymphatic drainage.

Passive targeting of calicheamicin conjugates yields therapeutic benefit in a variety of human tumors. The molecular characteristics of the IgG molecule and the use of an acid labile linker can be importance to allow efficacy by passive targeting. Calicheamicin conjugates designed for passive targeting may prove to be clinical assets in targeted delivery when tumors do not express tumor associated antigen or when extratumoral expression of these antigens prevents the use of actively targeted calicheamicin conjugate.

The therapeutic agents suitable for use in the present invention are cytotoxic drugs that inhibit or disrupt tubulin polymerization, alkylating agents that bind to and disrupt DNA, and agents which inhibit protein synthesis or essential cellular proteins such as protein kinases, enzymes and cyclins. Examples of such cytotoxic drugs include, but are not limited to thiotepa, taxanes, vincristine, daunorubicin, doxorubicin, epirubicin, actinomycin, authramycin, azaserines, bleomycins, tamoxifen, idarubicin, dolastatins/auristatins, hemiasterlins, esperamicins and maytansinoids.

Preferred cytotoxic drugs are the calicheamicins, which are an example of the methyl trisulfide antitumor antibiotics. As discussed previously, calicheamicin refers to a family of antibacterial and antitumor agents, as described in U.S. Pat. No. 4,970,198 (see also U.S. Pat. No. 5,108,912, both of which are incorporated herein in their entirety). In one preferred embodiment of the present process, the calicheamicin is an N-acyl derivative of calicheamicin or a disulfide analog of calicheamicin. The dihydro derivatives of these compounds are described in U.S. Pat. No. 5,037,651 and the N-acylated derivatives are described in U.S. Pat. No. 5,079,233, both of which are incorported in their entirety herein. Related compounds, which are also useful in this invention, include the esperamicins, described in U.S. Pat. Nos. 4,675,187; 4,539,203; 4,554,162; and 4,837,206, all of which are herein incorporated in their entirety. All of these compounds contain a methyltrisulfide that can be reacted with appropriate thiols to form disulfides, at the same time introducing a functional group such as a hydrazide or similar nucleophile. Two compounds that are useful in the present invention are disclosed in U.S. Pat. No. 5,053,394, and are shown in Table 1 of U.S. Pat. No. 5,877,296, gamma dimethyl hydrazide and N-acetyl gamma dimethyl hydrazide. All information in the above-mentioned patent citations is incorporated herein by reference.

Preferably, in the context of the present invention, the calicheamicin is N-acetyl gamma calicheamicin dimethyl hydrazide (N-acetyl calicheamicin DMH). N-acetyl calicheamicin DMH is at least 10- to 100-fold more potent than the majority of cytotoxic chemotherapeutic agents in current use. Its high potency makes it an ideal candidate for antibody-targeted therapy, thereby maximizing antitumor activity while reducing nonspecific exposure of normal organs and tissues.

Thus, in one embodiment, the conjugates of the present invention have the formula:
Pr(—X—W)ₘ

wherein:

Pr is an antibody;

X is a linker that comprises a product of any reactive group that can react with the antibody;

W is a cytotoxic drug from the calicheamicin family;

m is the average loading for a purified conjugation product such that the calicheamicin constitutes 3-9% of the conjugate by weight; and

(—X—W)ₘ is a cytotoxic drug derivative

Preferably, X has the formula
(CO-Alk¹-Sp¹-Ar-Sp²-Alk²-C(Z¹)=Q-Sp)
wherein

Alk¹ and Alk² are independently a bond or branched or unbranched (C₁-C₁₀) alkylene chain;

Sp¹ is a bond, —S—, —O—, —CONH—, —NHCO—, —NR—, —N(CH₂CH₂)₂N—, or —X—Ar—Y—(CH₂)ₙ-Z wherein X, Y, and Z are independently a bond, —NR—, —S—, or —O—, with the proviso that when n=0, then at least one of Y and Z must be a bond and Ar is 1,2-, 1,3-, or 1 ,4-phenylene optionally substituted with one, two, or three groups of (C₁-C₅) alkyl, (C₁-C₄) alkoxy, (C₁-C₄) thioalkoxy, halogen, nitro, —COOR, —CONHR, —(CH₂)ₙCOOR, —S(CH₂)ₙCOOR, —O(CH₂)ₙCONHR, or —S(CH₂)ₙCONHR, with the proviso that when Alk¹ is a bond, Sp¹ is a bond;

n is an integer from 0 to 5;

R is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt;

Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR wherein n and R are as hereinbefore defined or a 1,2-, 1,3-, 1,4-, 1,5-, 1,6-, 1,7-, 1,8-, 2,3-, 2,6-, or 2,7-naphthylidene or

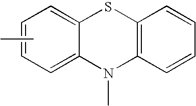

with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, or —S(CH₂)ₙCONHR wherein n and R are as defined above, with the proviso that when Ar is phenothiazine, Sp¹ is a bond only connected to nitrogen;

Sp² is a bond, —S—, or —O—, with the proviso that when Alk² is a bond, Sp² is a bond;

Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR, —ONHR, —O(CH₂)ₙCOOR, —S(CH₂)ₙCOOR, —O(CH₂)ₙCONHR, or —S(CH₂)ₙCONHR wherein n and R are as defined above;

Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₁₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp can be additionally substituted by lower (C₁-C₅) dialkylamino, lower (C₁-C₅) alkoxy, hydroxy, or lower (C₁-C₅) alkylthio groups; and

Q is =NHNCO—, =NHNCS—, =NHNCONH—, =NHNCSNH—, or =NHO—.

Preferably, Alk¹ is a branched or unbranched (C₁-C₁₀) alkylene chain; Sp is a bond, —S—, —O—, —CONH—, —NHCO—, or —NR wherein R is as hereinbefore defined, with the proviso that when Alk¹ is a bond, Sp¹ is a bond;

Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR wherein n and R are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR.

Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR.

Alk² and Sp² are together a bond.

Sp and Q are as immediately defined above.

In one embodiment, the conjugates of the present invention utilize the cytotoxic drug calicheamicin derivatized with a linker that includes any reactive group which reacts with an antibody, which is used as a proteinaceous carrier targeting agent to form a cytotoxic drug derivative-antibody conjugate. U.S. Pat. Nos. 5,773,001; 5,739,116 and 5,877,296, incorporated herein in their entirety, discloses linkers that can be used with nucleophilic derivatives, particularly hydrazides and related nucleophiles, prepared from the calicheamicins. These linkers are especially useful in those cases where better activity is obtained when the linkage formed between the drug and the linker is hydrolyzable. These linkers contain two functional groups. One group typically is a carboxylic acid that is utilized to react with the carrier. The acid functional group, when properly activated, can form an amide linkage with a free amine group of the carrier, such as, for example, the amine in the side chain of a lysine of an antibody or other proteinaceous carrier. The other functional group commonly is a carbonyl group, i.e., an aldehyde or a ketone, which will react with the appropriately modified therapeutic agent. The carbonyl groups can react with a hydrazide group on the drug to form a hydrazone linkage. This linkage is hydrolyzable (specifically, the linker is acid labile), allowing for release of the therapeutic agent from the conjugate after binding to the target cells. Preferably, the hydrolyzable linker is 4-(4-acetylphenoxy)butanoic acid (AcBut) or (3-Acetylphenyl)acetic acid (AcPAc).

Apart from the carrier function of the immunoglobulin, the use of an acid labile linker is relevant to the efficacy of the calicheamicin conjugate. While not wishing to be bound by any particular theory or mechanism of action, after accumulation of the calicheamicin conjugate in the tumor, the pericellular acidic environment may be responsible for the release of calicheamicin. This mechanism may be related to the fact that oncolytic effects of the calicheamicin conjugate in vivo were congruent with the sensitivity of tumor cells to calicheamicin in vitro. In addition, pinocytosis may also be related to a mechanism for incorporation of the calicheamicin conjugate. However, since an acid stabile linker was ineffective in the absence of a targeted antigen, this contribution may be less relevant.

The antibodies of the present invention are non-specific antibodies. Such antibodies are specific for an antigen that is not present on the tumor cells to which the cytotoxic conjugate is adminstered. Any known method can be used to determine the presence or absence of an antigen from the tumor cells, such as FACS or BIAcore analysis, for example. By substituting immunoglobulin in the conjugate by other macromolecules, carrier characteristics were identified underlying the therapeutic activity of a calicheamicin conjugate. Examples of carriers generally suitable for active targeting are liposomes, albumine, dextran or Poly Ethylene Glycol (PEG) polymers. Consistent with the finding that accumulation of immunoglobulin in grafted tumors was more pronounced than the accumulation of albumin, the antibody could neither be replaced by albumin nor by PEGylated Fc fragments without reduction or the loss of efficacy of the conjugate.

Examples of antibodies that may be used in the present invention include monoclonal antibodies (mAbs), for example, chimeric antibodies, humanized antibodies, primatized antibodies, resurfaced antibodies, human antibodies and biologically active fragments thereof, regardless of specificity, isotype or isoelectric point. The term antibody, as used herein, unless indicated otherwise, is used broadly to refer to both antibody molecules and a variety of antibody derived molecules. Such antibody-derived molecules comprise at least one variable region (either a heavy chain or light chain variable region) and include molecules such as Fab fragments, F(ab′)₂ fragments, Fd fragments, Fabc fragments, Sc antibodies (single chain antibodies), diabodies, individual antibody light single chains, individual antibody heavy chains, chimeric fusions between antibody chains and other molecules, and the like.

Preferably, the antibodies used in the present invention are a compete immunoglobulin having two heavy and two light chains. For example, the molecular mass and the general protein structure of the IgG molecule may be necessary to target sufficient amounts of calicheamicin to the tumor without causing lethality in the mice.

The antibodies of the present invention can be specific for any TAA, including, for example, CD22, CD33, HER2/neu; EGFR; PSMA; PSCA; MIRACL-26457; CEA; Lewis Y (Le^{y}) or 5T4. Exemplary antibodies include hp67.6 and g5/44, which are humanized IgG4 antibodies that specifically recognize human CD33 or CD22, respectively (see U.S. Pat. No. 5,773,001 and U.S Application Nos. 2004/0082764 A1 and 2004/0192900 A1, which are incorporated herein in their entirety). RITUXAN (rituximab) (IDEC Pharmaceuticals Corporation and Genentech), which is a chimeric IgG1-k antibody that recognizes CD20, is also an exemplary antibody. Another example is an anti-Lewis Y antibody designated hu3S193 (see U.S. Pat. Nos. 6,310,185; 6,518,415; 5,874,060, which are incorporated herein in their entirety) or, alternatively, G193, which is described in co-pending application entitled “Calicheamicin Conjugates” (AM101462). As TAAs are rarely exclusive products of tumor cells and expression of these antigens (e.g. Le^{y}, EGFR, or Her2/neu) in normal tissues can pose therapeutic dose limiting toxicity for calicheamicin conjugates that recognize these antigens, using a calicheamicin conjugate with a carrier antibody that fails to recognize any human antigen could bypass this problem.

The antibodies of the subject invention may be produced by a variety of methods useful for the production of polypeptides, e.g., in vitro synthesis, recombinant DNA production, and the like. Preferably, the antibodies are produced by recombinant DNA technology and protein expression methods. Techniques for manipulating DNA (e.g., polynucleotides) are well known to the person of ordinary skill in the art of molecular biology. Examples of such well-known techniques can be found in Molecular Cloning: A Laboratory Manual 2^{nd} Edition, Sambrook et al, Cold Spring Harbor, N.Y. (1989). Techniques for the recombinant expression of immunoglobulins, including humanized immunoglobulins, can also be found, among other places in Goeddel et al, Gene Expression Technology Methods in Enzymology, Vol. 185, Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993). Examples of conventional molecular biology techniques include, but are not limited to, in vitro ligation, restriction endonuclease digestion, PCR, cellular transformation, hybridization, electrophoresis, DNA sequencing, and the like.

The general methods for construction of vectors, transfection of cells to produce host cells, culture of cells to produce antibodies are all conventional molecular biology methods. Likewise, once produced, the recombinant antibodies can be purified by standard procedures of the art, including cross-flow filtration, ammonium sulphate precipitation, affinity column chromatography, gel electrophoresis, diafiltration and the like. The host cells used to express the recombinant antibody may be either a bacterial cell, such as E. coli, or preferably, a eukaryotic cell. Preferably, a mammalian cell such as a PER.C.6 cell or a Chinese hamster ovary cell (CHO) is used. The choice of expression vector is dependent upon the choice of host cell, and is selected so as to have the desired expression and regulatory characteristics in the selected host cell.

Preferably, the conjugates used in the present methods maintain the binding kinetics and specificity of the naked antibody. Any known method can be used to determine the binding kinetics and specificty of the conjugate, such as FACS or BIAcore analysis, for example.

The non-specific antibodies can be used in conjunction with, or attached to other antibodies (or parts thereof) such as human or humanized monoclonal antibodies. These other antibodies may be reactive with other markers (epitopes) characteristic for the disease against which the antibodies of the invention are directed or may have different specificities chosen, for example, to recruit molecules or cells of the human immune system to the diseased cells. The antibodies of the invention (or parts thereof) may be administered with such antibodies (or parts thereof) as separately administered compositions or as a single composition with the two agents linked by conventional chemical or by molecular biological methods. Additionally, the diagnostic and therapeutic value of the antibodies of the invention may be augmented by labeling the humanized antibodies with labels that produce a detectable signal (either in vitro or in vivo) or with a label having a therapeutic property. Some labels, e.g., radionuclides may produce a detectable signal and have a therapeutic property. Examples of radionuclide labels include ¹²⁵I, ¹³¹I, ¹⁴C. Examples of other detectable labels include a fluorescent chromophore, such as fluorescein, phycobiliprotein or tetraethyl rhodamine for fluorescence microscopy, an enzyme which produces a fluorescent or colored product for detection by fluorescence, absorbance visible color or agglutination, which produces an electron dense product for demonstration by electron microscopy; or an electron dense molecule such as ferritin, peroxidase or gold beads for direct or indirect electron microscopic visualization. Labels having therapeutic properties include drugs for the treatment of cancer, such as methotrexate and the like.

The conjugates used in the present methods may be the sole active ingredient in the therapeutic or diagnostic composition/formulation or may be accompanied by other active ingredients (e.g., chemotherapy agents, hormone therapy agents, or biological therapy agents described below), including other antibody ingredients, for example, anti-CD19, anti-CD20, anti-CD33, anti-T cell, anti-IFNγ or anti-LPS antibodies, or non-antibody ingredients such as cytokines, growth factors, hormones, anti-hormones, cytotoxic drugs and xanthines.

These compositions/formulations can be administered to patients for treatment of cancer. According to the present invention, a therapeutically effective amount of a non-specific antibody conjugated to a cytotoxin is administered to a patient in need thereof. Alternatively, the compostition or formulation is used to manufacture a medicament for treatment of cancer. It should be appreciated that this method or medicament can be used to treat any patient with cancer cells that do not express the antigen to which the non-specific antibody binds. There may, however, be a correlation between efficacy of treatment and sensitivity of the cancer cells to calicheamicin. In one embodiment, the cancer treated is gastric carcinoma, colon carcinoma, non-small cell lung carcinoma (NSCLC), breast carcinoma, epidermoid carcinoma, or prostate carcinoma.

The present treatment methods can be used in combination with other cancer treatments, including surgery, radiation, chemotherapy, hormone therapy, biologic therapies, bone marrow transplantation (for leukemias and other cancers where very high doses of chemotherapy are needed). New treatments are also currently being developed and approved based on an increased understanding of the biology of cancer.

Two general classes of radiation therapy exist and can be used in the present methods. In one class, brachytherapy, direct implants of a radioisotope are made into the tumor to deliver a concentrated dose to that area. In the other class, teletherapy, a beam is used to deliver radiation to a large area of the body or to the whole body in total body irradiation (TBI).

Any suitable chemothepeutic agent can be used in the present methods. These chemotherapeutic agents generally fall into the following classes (with examples of each): antimetabolites (e.g., folic acid antagonists such as methotrexate, purine antagonists such as 6-mercaptopurine (6-MP), and pyrimidine antagonists such as 5-fluorouracil (5-FU)); alkylating agents (cyclophosphamide); DNA binding agents (cisplatin or oxaliplatin); anti-tumor antibiotics (doxorubicin or mitoxantrone); mitotic inhibitors (e.g., the taxanes or microtubule inhibitors such as vincristine) or topoisomerase inhibitors (camptothecan or taxol). More specific examples are described below.

Hormone therapies relevant to the present methods include, for example, corticosteroids for leukemias and myelomas, estrogens and anti-estrogens for breast cancers, and androgens and anti-androgens for prostate cancer.

Biologic therapy uses substances derived from the body. Examples of suitable therapies in the present methods include antibodies (e.g., anti-EGFR antibodies, such as cetuximab or trastuzumab, or anti-VEGF antibodies, such as bevacizumab), T-cell therapies, interferons, interleukins, and hematopoietic growth factors.

Bone marrow transplantation can be used for treatment of some cancers, notably leukemias. To treat leukemias, the patient's marrow cells are destroyed by chemotherapy or radiation treatment. Bone marrow from a donor that has matching or nearly matching HLA antigens on the cell surface is then introduced into the patient. Bone marrow transplantation is also used to replace marrow in patients who required very high doses of radiation or chemotherapy to kill the tumor cells. Transplants are classified based on donor source. In allogeneic transplants, the marrow donor is often not genetically related but has matches with at least five out of six cell surface antigens that are the major proteins recognized by the immune system (HLA antigens). In autologous transplantation, patients receive their own marrow back after chemotherapy or radiation treatment. This type of bone marrow transplant can be used for non-marrow related cancers for which conventional treatment doses have been incompletely effective.

Additionally, new emerging approaches that can be used in the present methods, some of which are approved or in clinical trials, are being developed based on an increased understanding of the molecular and cellular bases of cancer and the progression of the disease. Protein kinase inhibitors (both small molecules and antibodies) that inhibit the phosphorylation cascade can be used (e.g., erlotinib or imatinib mesylate). Any antimetastasis agent can be used that blocks the spread of cancer cells and the invasion of new tissues. Antiangiogensis agents can be used that block development of blood vessels that nourish a tumor (e.g, thalidomide). Other agents that can be used are antisense oligonucleotides, which block production of aberrant proteins that cause proliferation of tumor cells. Gene therapy can also be used to introduce genes into T cells that are injected into the patient and are designed to kill specific tumor cells. Also, p53 can be targeted by introducing normal p53 genes into mutant cancer cells, for example, to re-establish sensitivity to chemotherapeutic drugs.

In one embodiment, the compositions/formulations of the present invention are used in combination with bioactive agents. Bioactive agents commonly used include antibodies, growth factors, hormones, cytokines, anti-hormones, xanthines, interleukins, interferons, cytotoxic drugs and antiangiogenic proteins.

Bioactive cytotoxic drugs commonly used to treat proliferative disorders such as cancer, and which may be used together with the calicheamicin—anti-Lewis Y antibody conjugates include: anthracyclines such as doxorubicin, daunorubicin, idarubicin, aclarubicin, zorubicin, mitoxantrone, epirubicin, carubicin, nogalamycin, menogaril, pitarubicin, and valrubicin for up to three days; pyrimidine or purine nucleosides such as cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin; alkylating agents such as cyclophosphamide, melphalan, thiotepa, ifosfamide, carmustine, cisplatin, CKD-602, ledoxantrone, rubitecan, topotecan hydrochloride, LE-SN38, afeletecan hydrochloride, XR-11576 and XR-11612; antimetabolites such as methotrexate, 5 flurouracil, tegafur/uracil (UFT), ralititrexed, capecitabine, leucovorin/UFT, S-1, pemetrexed disodium, tezacitabine, trimetrexate glucuronate, thymectacin, decitabine; antitumor antibodies such as edrecolomab, mitomycin, mitomycin C and oxaliplatin; vinca alkyloids such as vincristine, vinblastine, vinorelbine, anhydrovinblastine; angiogenesis inhibitors such as vatalanib succinate, oglufanide, RPI-4610; signal transduction inhibitors such as gefitinib, 317615.2 HCL, indisulam, lapatinib, sorafenib, WHI-P131; apoptosis inducers such as alvocidib hydrochloride, irofulven, sodium phenylbutyrate, bortezomib, exisulind, MS-2167; epipodophyllotoxins such as etoposide; and taxanes such as paclitaxel, doceltaxel, DHA-paclitaxel, ixabepilone, polyglutamate paclitaxel, or epothilones.

Other chemotherapeutic/antineoplastic agents that may be administered in combination with hu3S193-AcBut-CM or CMD-193 or AG G193-AcBut-CM include adriamycin, cisplatin, carboplatin, cyclophosphamide, dacarbazine, ifosfamide, vindesine, gemcitabine, edatrexate, irinotecan, mechlorethamine, altretamine, carboplatine, teniposide, topotecan, gemcitabine, thiotepa, fluxuridine (FUDR), MeCCNU, vinblastine, vincristine, mitoxantrone, bleomycin, mechlorethamine, prednisone, procarbazine methotrexate, flurouracils, etoposide, taxol and its various analogs, mitomycin, thalidomide and its various analogs, GBC-590, troxacitabine, ZYC-300, TAU, (R) flurbiprofen, histamine hydrochloride, tariquidar, davanat-1, ONT-093. Administration may be concurrently with one or more of these therapeutic agents or, alternatively, sequentially with one or more of these therapeutic agents.

Bioactive antibodies that can be administered with the antibody conjugates of this invention include, but are not limited to Herceptin, Zevalin, Bexxar, Campath, cetuximab, bevacizumab, ABX-EGF, MDX-210, pertuzumab, trastuzumab, I-131 ch-TNT-1/b, hLM609, 6H9, CEA-Cide Y90, IMC-1C11, ING-1, sibrotuzumab, TRAIL-R1 Mab, YMB-1003, 2C5, givarex and MH-1.

The calicheamicin—anti-Lewis Y antibody conjugates may also be administered alone, concurrently, or sequentially with a combination of other bioactive agents such as growth factors, cytokines, steroids, antibodies such as anti-Lewis Y antibody, rituximab and chemotherapeutic agents as a part of a treatment regimen. Calicheamicin—anti-Lewis Y antibody conjugates may also be administered alone, concurrently, or sequentially with any of the above identified therapy regimens as a part of induction therapy phase, a consolidation therapy phase and a maintenance therapy phase.

The conjugates of the present invention may also be administered together with other bioactive and chemotherapeutic agents as a part of combination chemotherapy regimen for the treatment of relapsed aggressive carcinoma. Such a treatment regimen includes: CAP (Cyclophosphamide, Doxorubicin, Cisplatin), PV (Cisplatin, Vinblastine or vindesine), CE (Carboplatin, Etoposide), EP (Etoposide, Cisplatin), MVP (Mitomycin, Vinblastine or Vindesine, Cisplatin), PFL (Cisplatin, 5-Flurouracil, Leucovorin), IM (Ifosfamide, Mitomycin), IE (Ifosfamide, Etoposide); IP (Ifosfamide, Cisplatin); MIP (Mitomycin, Ifosfamide, Cisplatin), ICE (Ifosfamide, Carboplatin, Etoposide); PIE (Cisplatin, Ifosfamide, Etoposide); Viorelbine and Cisplatin; Carboplatin and Paclitaxel; CAV (Cyclophosphamide, Doxorubicin, Vincristine), CAE (Cyclophosphamide, Doxorubicin, Etoposide); CAVE (Cyclophosphamide, Doxorubicin, Vincristine, Etoposide); EP (Etoposide, Cisplatin); CMCcV (Cyclophosphamide, Methotrexate, Lomustine, Vincristine); CMF (Cyclophosphamide, Methotrexate, 5-Flurouracil); CAF (Cyclophosphamide, Doxorubicin, 5-Flurouracil); CEF (Cyclophosphamide, Epirubicin, 5-Flurouracil); CMFVP (Cyclophosphamide, Methotrexate, 5-Flurouracil, Vincristine, Prednisone); AC (Doxorubicin, Cyclophosphamide); VAT (Vinblastine, Doxorubicin, Thiotepa); VATH (Vinblastine Doxorubicin, Thiotepa, Fluosymesterone); CDDP+VP-16 (Cisplatin, Etoposide, Mitomycin C+Vinblastine).

It should be appreciated that, in the context of the present invention, treating means inhibiting, preventing, or slowing cancer growth, including delayed tumor growth and inhibition of metastasis.

The compositions/formulations of the present invention can be administered as a second-line monotherapy. By second-line is meant that the present compositions/formulations are used after treatment with a different anti-cancer treatment, examples of which are described above. Alternatively, the compositions or formulations can be administered as a first-line combination therapy with another anti-cancer treatment described above.

The antibody compositions may be administered to a patient in a variety of ways. Direct delivery of the compositions will generally be accomplished by injection, subcutaneously, intraperitoneally, intravenously or intramuscularly, or delivered to the interstitial space of a tissue. Preferably, the pharmaceutical compositions may be administered parenterally, i.e., subcutaneously, intramuscularly or intravenously. The compositions can also be administered into a lesion. Dosage treatment may be a single dose schedule or a multiple dose schedule.

Passive targeting of calicheamicin may be less efficacious than active targeting. This relative difference manifests itself by shorter duration of the tumor remission and the higher doses necessary to obtain efficacy with a calicheamicin conjugate that uses a passive targeting mechanism. Yet, a passive targeting strategy may in certain circumstances be indicated because it bypasses the need for homogenous expression or overexpression of a tumor-associated antigen. However, the maximum tolerated dose of a calicheamicin conjugate designed for passive targeting may be higher than for an active targeting calicheamicin conjugate.

A variety of aqueous carriers can be used, e.g., water, buffered water, 0.4% saline, 0.3% glycine and the like. These solutions are sterile and generally free of particulate matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate. The concentration of antibody in these formulations can vary widely, e.g., from less than about 0.5%, usually at or at least about 1% to as much as 15 or 20% by weight and will be selected primarily based on fluid volumes and viscosities, for example, in accordance with the particular mode of administration selected.

The methods of the present invention involve administration of a therapeutically effective amount of a conjugate. The term therapeutically effective amount as used herein refers to an amount of a therapeutic agent needed to treat, ameliorate or prevent a targeted disease or condition, or to exhibit a detectable therapeutic or preventative effect. For any conjugate, the therapeutically effective dose can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise effective amount for a human subject will also depend upon the nature and severity of the disease state, the general health of the subject, the age, weight and gender of the subject, diet, time and frequency of administration, drug combination(s), reaction sensitivities and tolerance/response to therapy. If the conjugate is being used prophylactically to treat an existing condition, this will also affect the effective amount. This amount can be determined by routine experimentation and is within the judgment of the clinician. Generally, an effective dose will be from 0.01 mg/m² to 50 mg/m², preferably 0.1 mg/m² to 20 mg/m², more preferably about 10-15 mg/m², calculated on the basis of the proteinaceous carrier.

The frequency of dose will depend on the half-life of the conjugate and the duration of its effect. If the conjugate has a short half-life (e.g., 2 to 10 hours) it may be necessary to give one or more doses per day. Alternatively, if the conjugate molecule has a long half-life (e.g., 2 to 15 days) it may only be necessary to give a dosage once per day, once per week or even once every 1 or 2 months.

A composition can also contain a pharmaceutically acceptable carrier for administration of the antibody conjugate. A pharmaceutical carrier can be any compatible, non-toxic substance suitable for delivery of the monoclonal antibodies to the patient. Sterile water, alcohol, fats, waxes, and inert solids may be included in the carrier. The carrier should not itself induce the production of antibodies harmful to the individual receiving the composition and should not be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. Pharmaceutically accepted adjuvants (buffering agents, dispersing agent) may also be incorporated into the pharmaceutical composition.

Pharmaceutically acceptable salts can be used, for example, mineral acid salts, such as hydrochlorides, hydrobromides, phosphates and sulfates, or salts of organic acids, such as acetates, propionates, malonates and benzoates.

Pharmaceutically acceptable carriers in therapeutic compositions/formulations may additionally contain liquids such as water, saline, glycerol, and ethanol. Auxiliary substances, such as wetting or emulsifying agents or pH buffering substances, may be present in such compositions. Such carriers enable the compositions to be formulated as tablets, pills, dragees, capsules, liquids, gels, syrups, slurries and suspensions, for ingestion by the patient.

Preferred forms for administration include forms suitable for parenteral administration, e.g., by injection or infusion, for example, by bolus injection or continuous infusion. Where the product is for injection or infusion, it may take the form of a suspension, solution or emulsion in an oily or aqueous vehicle and it may contain formulatory agents, such as suspending, preserving, stabilizing and/or dispersing agents.

Although the stability of the buffered conjugate solutions is adequate for a short time, long-term stability is poor. To enhance stability of the conjugate and to increase its shelf life, the antibody-drug conjugate may be lyophilized to a dry form, for reconstitution before use with an appropriate sterile liquid. The problems associated with lyophilization of a protein solution are well documented. Loss of secondary, tertiary and quaternary structure can occur during freezing and drying processes. Contacting them with a cryoprotectant, a surfactant, a buffering agent, and an electrolyte in a solution and then lyophilizing the solution can preserve biological activity of these compositions/formulations. A lyoprotectant also can be added to the solution.

The conjugates can be administered by any number of routes including, but not limited to, oral, intravenous, intramuscular, intra-arterial, intramedullarly, intrathecal, intraventricular, transdermal, transcutaneous (see PCT Publication No.: WO98/20734), subcutaneous, intraperitoneal, intranasal, enteral, topical, sublingual, intravaginal or rectal routes. Hyposprays may also be used to administer the compositions of the invention. Typically, the compositions may be prepared as injectables, either as liquid solutions or suspensions. Solid forms suitable for solution in, or suspension in, liquid vehicles prior to injection may also be prepared.

### EXAMPLES

### Example 1

### Materials and Methods

Calicheamicin Conjugates

Calicheamicin analogues were conjugated to various carrier molecules with either acid labile or acid stabile linkers. The acid labile 4-(4′-acetylphenoxy)butanoic acid (AcBut) or (3-Acetylphenyl)acetic acid (AcPAc) allow for acid hydrolysis of the hydrazone group and for disulfide reduction in the lysosomes. The acid stabile 4-mercapto-4-methyl-pentanoic acid (Amide) allows only for dissociation at the disulfide group. The calicheamicin analoges, N-acetyl-γ-calicheamicin dimethyl hydrazide (CalichDMH) or N-acetyl-γ-calicheamicin dimethyl acid (CalichDMA) were conjugated with acid labile or acid stable linkers, respectively.

Cells and Culturing Conditions

N87 (CRL-5822), HT29 (HTB-38), LOVO (CCL-229), A431 (CRL-1555) and LNCaP (CRL-1740) were purchased from the American Type Culture Collection (ATCC). Cell lines obtained from ATCC were maintained in culture medium as specified in the ATCC-catalogue. L2987 was a gift from Dr. C. Siegall (Seattle Genetics, Bothell, Wash.). These cells were grown in RPMI 1640 supplemented with 10% FBS, 2 mM gln, 100 IU penicillin and 100 μg streptomycin (hereafter called pen/strep) and 0.05 mg gentamycin. PC14PE6, PC3MM2 and MDAMB435 were obtained from Dr. I. Fidler (MD Anderson, Houston, Tex.). PC14PE6 and PC3MM2 were maintained in minimum essential medium supplemented with 10% v/v FBS, 2 mM gln, 1 mM sodium pyruvate, 0.2 mM non-essential amino acids, 2% MEM vitamin solution, and pen/strep. MDAMB435/5T4 are MDAMB435 cells that were transfected with a plasmid encoding the oncofetal protein, 5T4, and the neomycin resistance marker. These cells were cultured in minimum essential medium with Earle's salts supplemented with 10% v/v FBS, 2 mM gln, 1 mM sodium pyruvate, 0.2 mM non-essential amino acids, 2% MEM vitamin solution, and 50% pen/strep and 1.5 mg/ml G418. Dr. Scott A. (Ludwig Institute for Cancer Research, Melbourne, Australia) provided A431/Le^{y} cells that are Lewis Y positive variants of A431. They were cultured in DMEM/F12 supplemented with 10% v/v FBS, 2 mM gin and pen/strep. KB 8.5 cells were obtained from Dr. Shen and cultured in DMEM (high glucose) supplemented with 20% v/v FBS 2 mM gin, 10 μM sodium pyruvate, 10% pen/strep and 0.25 mM colchicines.

Antibodies and Conjugates

Hp67.6 and g5/44 are humanized IgG4 antibodies that specifically recognize human CD33 or CD22, respectively. Rituxan (IDEC Pharmaceuticals Corporation and Genentech) is a chimeric IgG1-κ antibody that recognizes CD20. MOPC is monoclonal IgG1-κ mouse antibody with unknown specificity that is commonly used as negative control in immunodetection methods.

For FACS-analysis, human IgG (huIgG, Zymed, San Francisco, Calif.) and mouse IgG and FITC-labeled goat anti-huIgG (FITC/α-huIgG, Zymed, San Francisco, Calif.) were used as control antibody and as secondary antibody, respectively. Conjugation of N, acetyl γ-calicheamicin dimethyl hydrazide (CalichDMH) was done by means of the acid labile AcBut (4-(4′-acetylphenoxy)butanoic acid) or AcPAc ((3-Acetylphenyl)acetic acid) linkers. Acid stabile conjugates were obtained by linking N, acetyl γ-calicheamicin dimethyl amide (CalichDMA) with an Amide (4-mercapto-4-methyl-pentanoic acid) linker to the antibodies. The molar ratio of calicheamicin to antibody showed a variation between 2:1 and 6:1 mol:mol. Processes for conjugating calicheamicin to antibodies are described in U.S. Pat. Nos. 5,773,001; 5,739,116; and 5,877,296 (all information in these patent citations is incorporated herein by reference).

Synthetic Macromolecules (FcPEGL AND FcPEGB)

(Fab)₂ fragments of hp67.6 were generated by digestion of 2.8 g antibody with 2.8 mg pepsin (Worthing Biochem.Corp., Freehold, N.J.) in 10 mM citrate buffer (pH 3.5, 37° C.) for 40 min and neutralized to pH 7 with K₂HPO₄. The digest was fractionated using a Macroprep high Q column (160 ml) chromatography in 10 mM Tris acetate pH 8. The (Fab)₂ eluted in the unbound fraction.

(Fab)₂ fragments of hp67.6 were then PEGylated. Twenty mg of (Fab)₂ was mixed with either 40 mg of linear 20 kDa PEG (N-Hydroxysuccinimidyl ester of Methoxy poly(ethylene glycol)propionic acid) or 60 mg of branched (10 kDa )₂ PEG (N-Hydroxysuccinimidyl ester of Methoxy poly(ethylene glycol)) in 10 mM potassium phosphate buffer pH 8.0. Both PEG stocks were made in water and used immediately. The reaction was allowed to proceed at 20° C. for 60 min.

Apparent MW was determined by SDS-PAGE and permeation chromatography. The average MW based on the elution position of the PEGylated (Fab)₂ is ˜250 kDa for the branched (10 kDa )₂ PEG and ˜300 kDa for the linear 20 kDa PEGylated (Fab)₂. SDS-PAGE indicated that the predominant species were (Fab)₂:PEG at a molar ratio of 1:2 and 1:3.

To PEGylate hp67.6, 50 mg of the antibody was mixed with 100 mg PEG (0.5 ml of 200 mg/ml of branched (10 kDa )₂ PEG stock) in 40 mM HEPES buffer pH 8.0, at a final protein concentration of 10.6 mg/ml. The reaction was allowed to proceed at 20° C. for 60 min.

FACS-Analysis

Aliquots of 10⁵ cells were suspended in 100 ul phosphate buffered saline supplemented with 1% v/v bovine serum albumin (PBS/BSA). The cells were then incubated at 4° C for 30 minutes in 10 μγ/ml primary antibody (hp67.7, hg544, Rituxan or MOPC) or conjugates of these antibodies as specified in the result section. Binding of the primary antibody to the cells was revealed by FITC/α-huIgG.

Determination of ED₅₀ In Vitro

A vital dye (MTS) staining was used to determine the number of surviving cells following exposure to various treatments. MTS (non-radioactive cell proliferation assay kit) was purchased from Promega (Madison, Wis.) and used according to the manufacturer's specifications. For each cell line a calibration curve (cell number versus optical density after 2 h) was established to estimate an appropriate initial seeding density. Cells were then seeded in 96-multiwell dishes at a density of 750 to 5,000 cells per well. Immediately after seeding, the cells were exposed to various concentrations (range 0 to 500 ng calicheamicin equivalents/ml) of hp67.6-AcBut-CalichDMH and CalichDMH. Following determination of the number of cells surviving 96 h of drug-exposure, the ED₅₀ was calculated based on the logistic regression parameters derived from the dose-response curves. The ED₅₀ was defined as the concentration of drug (ng/ml CalichDMH) that caused a 50% reduction of the cell number after 96 hours.

### Example 2

### Efficacy of HU3S193-DMH In Vivo

Subcutaneous tumors of N87, LOVO, A431/Le^{y}, LS174T and L2987 were grown in athymic nude mice (Charles River, Wilmington, Mass.). Two-month-old female mice were injected with respectively 5×10⁶ N87, LOVO, A431/Le^{y} or LS174T cells per mouse. L2987 cells were injected in male nude mice that were between 7 and 8 weeks old. To grow tumors, N87 cells had to be mixed (1:1, vol/vol) with MATRIGEL® (Collaborative Biomedical Products, Belford, Mass.) prior to injection. Two perpendicular diameters of the tumor were measured by means of calipers at time intervals specified in the result section. The tumor volume was calculated according to the formula of Attia&Weiss: A²×B×0.4. A and B are symbols for the smaller and the larger tumor diameter, respectively. The treatment schedules, dose and number of mice per group are specified in the result section and in the figure legends.

### Example 3

### In Vivo Distribution of ¹²⁵I-Labeled Conjugate

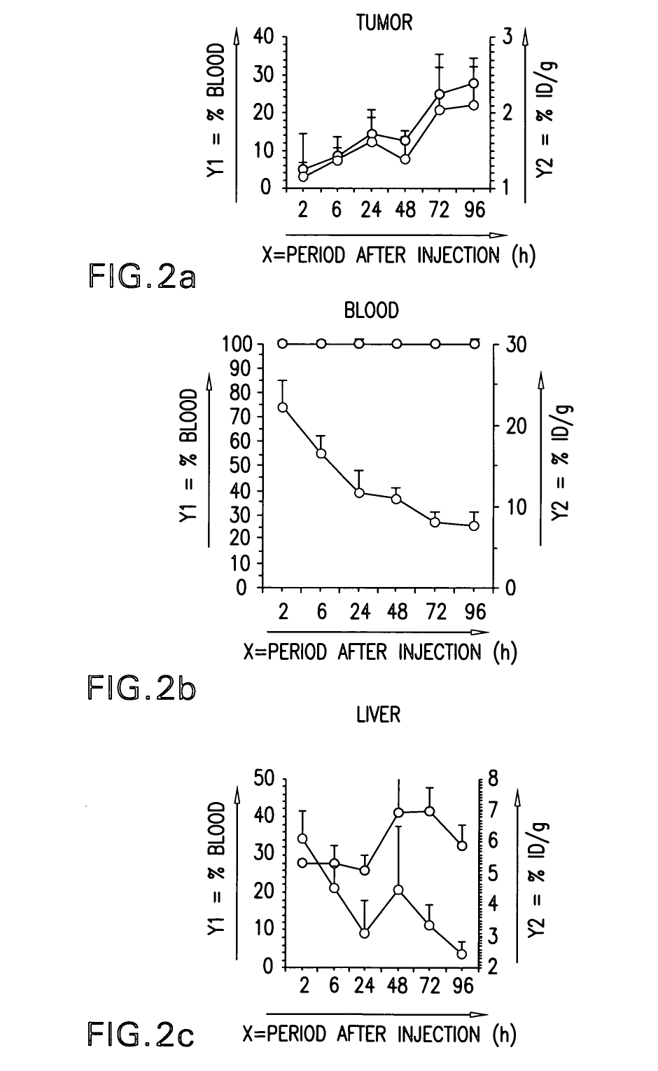

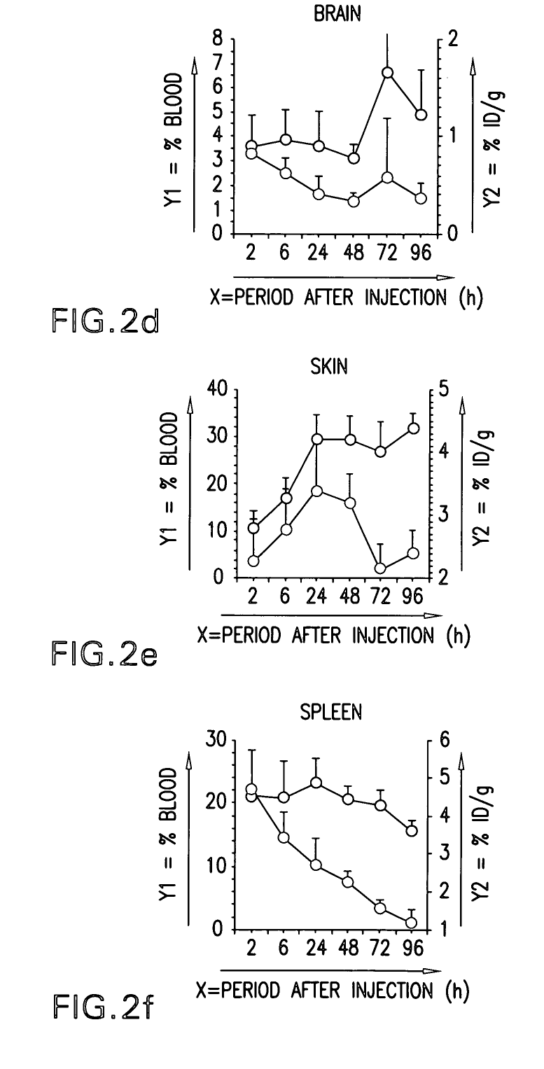

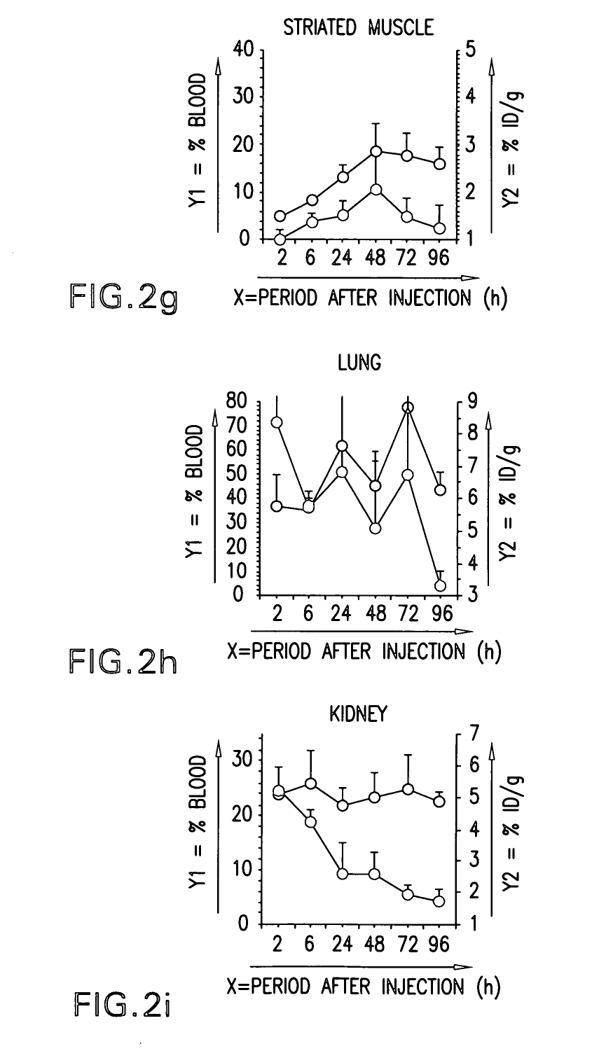

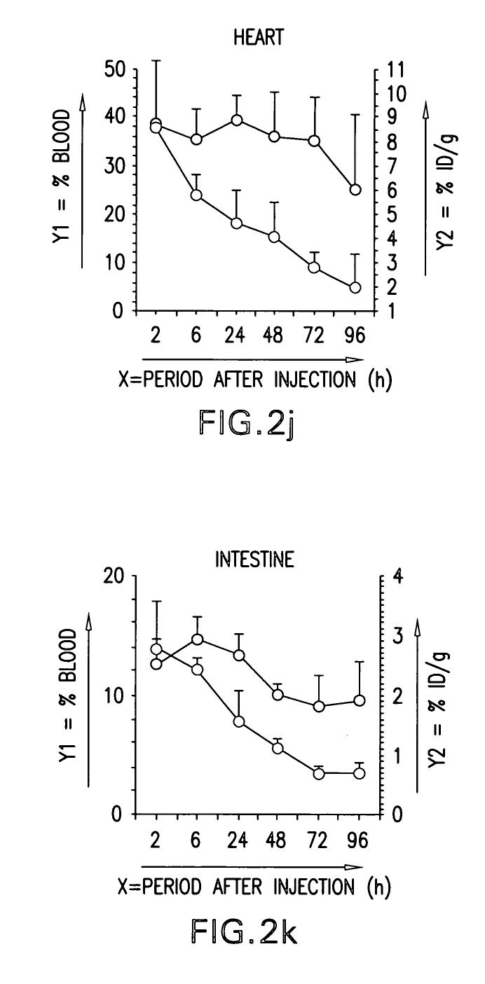

Gemtuzumab ozogamicin was labeled with ¹²⁵I using the Bolton-Hunter reagent (NEN, Boston, Mass.). A group of 30 tumor-bearing female nude mice were injected in the lateral tail vein with ¹²⁵I-labeled conjugate 20 μCi/200 mg. The tumor weight at the time of injection was approximately 1 g. Groups of 5 mice were killed by CO₂ inhalation at 2, 6, 24, 48, 72 and 96 h following the injection. The amount of γ radiation in the tissues as specified in FIG. 2 was determined at these time points. Biodistribution of the conjugate was expressed as a percentage of the injected dose per gram tissue (% ID/g) or as a percentage of the blood level at a given time point (% Blood). Steadily increasing concentrations of hp67.6-AcBut-CalichDMH were exclusively observed in tumor tissue. The doubling time of accumulation is 150 h for A431 tumors.

### Example 4

### Passive Targeting of hp67.6-AcBut-CalichDMH

Hp67.6-AcBut-CalichDMH inhibits growth of various subcutaneous xenografts despite undetectable amounts of the targeted antigen, CD33, on the tumor cells.

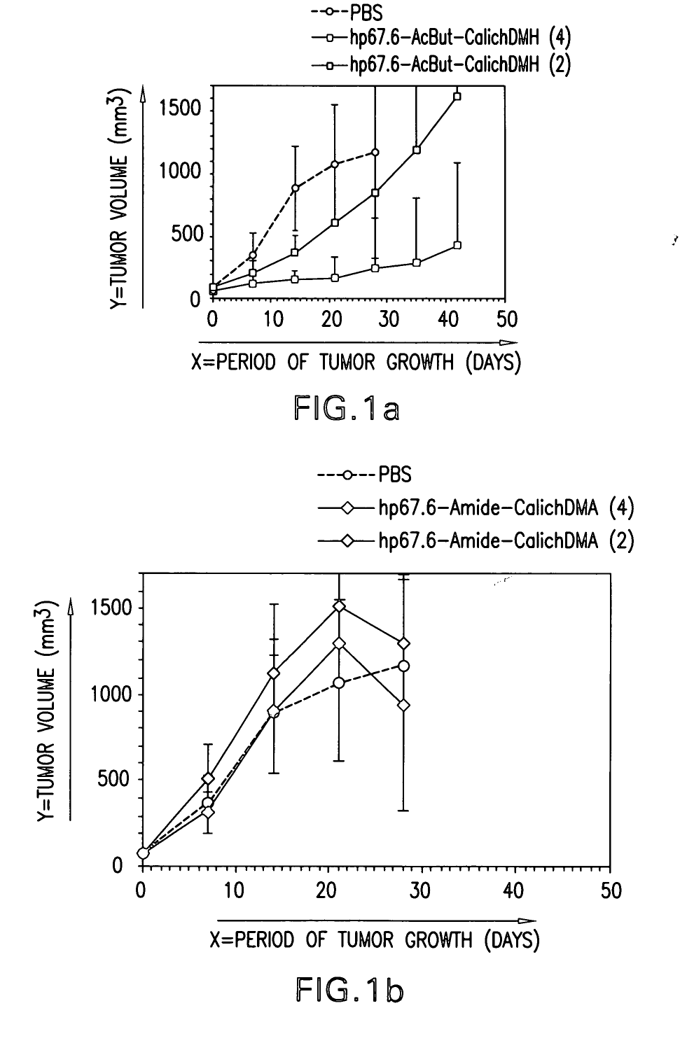

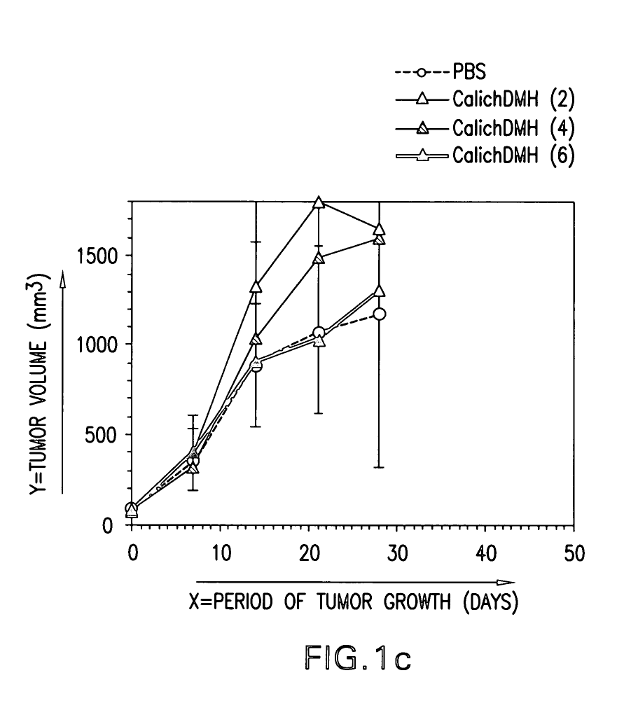

The oncolytic effect of hp67.6-AcBut-CalichDMH was demonstrated in multiple xenograft models. Table 1 (CD33-expression on carcinoma cells in vitro) lists the cell lines used to generate xenografts in nude mice and their expression of CD33 as measured by flow cytometry. The signal obtained using hp67.6 or hp67.6-AcBut-CalichDMH as primary antibody was mostly coinciding (reMCF approximates 1) with the signal obtained after using a negative control antibody, huIgG4. As illustrated in FIG. 1, hp67.6-AcBut-CalichDMH inhibits tumor growth of A431 epidermoid carcinoma xenografts notwithstanding the absence of CD33 on the cell membranes of these cells. All the groups of mice in the presented experiment were treated according to a regimen of 1 dose per mouse, given 3 times intraperitoneally with an interval of 4 days (Q4D×3). Mice with xenografts of approximately 80 mm³ were selected prior to treatment. The amounts of CalichDMH or conjugate given are expressed in calicheamicn equivalents. Up to 27 days following treatment, the growth of A431 xenografts was significantly (p=0.03) inhibited following administration of 3 doses of 4 μg hp67.6-AcBut-CalichDMH. Evaluation after 27 days was not possible since the tumor size in the control group became too large and necessitated killing of these mice for humane reasons.

TABLE 1

| Cell line | | reMCF hp67.6^{[a]} | | | reMCF hp67.6-AcBut-CalichDMH^{[d]} | | |
|---|---|---|---|---|---|---|---|
| designation | Tissue of origin | average | n^{[b]} | range^{[c]} | average | n | range |
| N87 | Gastric Carcinoma | 0.96 | 5 | 0.61-1.07 | 0.8 | 3 | 0.38-1.38 |
| HT29 | Colon Carcinoma | | | | | | |
| LOVO | Colon Carcinoma | 0.86 | 1 | | 0.91 | 1 | |
| PC14PE6 | NSCLC^{[e]} | | | | | | |
| L2987 | NSCLC | 0.75 | 1 | | 0.62 | 1 | |
| MDAMB435/5T4 | Breast Carcinoma | 0.48 | 4 | 0.42-0.56 | 0.74 | 2 | 0.60-0.87 |
| A431 | Epidermoid Carcinoma | 0.73 | 2 | 0.67-0.78 | 0.68 | 1 | |
| A431/Ley | Epidermoid Carcinoma | 0.56 | 1 | | 0.44 | 1 | |
| KB 8.5 | Epidermoid Carcinoma | 1.86 | 4 | 0.57-3.2 | 1 | 2 | 0.77-1.23 |
| LNCaP | Prostate Carcinoma | 1.07 | 2 | 0.48-1.65 | 0.81 | 2 | 0.46-1.15 |
| PC3MM2 | Prostate Carcinoma | 3.88 | 3 | 2.97-4.96 | 4.3 | 1 | |

^{[a]}= relative median channel fluorescence using hp67.6 as primary antibody
^{[b]}= number of independent determinations
^{[c]}= minimum and maximum of n determinations determinations
^{[d]}= relative median channel fluorescence using CMA-676 as primary antibody
^{[e]}= Non-Small Cell Lung Carcinoma

Binding of calicheamicin with an acid labile AcBut linker to hp67.6 yields an effective tumor inhibiting conjugate (FIG. 1A); however, substituting AcBut linker for an acid stabile Amide linker annihilates the efficacy of the conjugate (FIG. 1B) and administration of free calicheamicin does not cause inhibition of tumor growth (FIG. 1C). Specifically, xenografts treated with 2 μg/dose hp67.6-AcBut-CalichDMH only remained significantly (p=0.004) smaller than the controls for 21 days (FIG. 1A). Administration of hp67.6-Amide-CalichDMA or CalichDMH at equivalent or higher doses than hp67.6-AcBut-CalichDMH did not inhibit tumor growth (FIGS. 1B and 1C). The results presented in FIG. 1 demonstrate not only a significant inhibition of tumor growth by hp67.6-AcBut-CalichDMH but also dependence of this effect on the linker used for conjugation. Control CalichDMH is ineffective.

To determine if the efficacy of p67.6-AcBut-CalichDMH was related to a slow release of CalichDMH from the peritoneum, the experiment was repeated using the intravenous route for administration of the drugs while maintaining the same dose, frequency and interval of the treatments. Significant growth inhibition was observed following treatment with hp67.6-AcBut-CalichDMH. Twenty-seven days following onset of therapy, the average tumor sizes of mice treated with 4 or 2 μg of this conjugate were respectively 11 or 23% of the control tumors. Intravenous administration of hp67.6-Amide-CalichDMA or CalichDMH did not yield significant tumor growth inhibition.

As shown in Table 2 (tumor volume reduction of CD33-tumor xenografts following treatment (T) with hp67.6-AcBut-CalichDMH and expressed as a percentage of controls treated with vehicle (T/C %)), hp67.6-AcBut-CalichDMH inhibited tumor growth of human tumor xenografts with diverse histiotypic origin. Tumor growth inhibition is presented as a T/C-value. This value is the average tumor volume of a group of mice that were treated with hp67.6-AcBut-CalichDMH (T) expressed as a percentage of the average tumor volume of a control group (C). Both T and C are determined at the same day following initiation of treatment. The T/C-values in table II were derived from 27 independent experiments and were determined between 17 and 34 days after injection of the first dose of hp67.6-AcBut-CalichDMH. Despite variability in magnitude of the response, the data clearly demonstrate that hp67.6-AcBut-CalichDMH at a dose of 4 μg/mouse and a regimen of Q4D×3 significantly inhibits tumor growth in the majority of xenografts. Significant inhibition was also observed when lower amounts of the conjugate were administered.

TABLE 2

| | | hp67.6-AcBut- | | |
|---|---|---|---|---|
| | | Calich DMH | | |
| | | (amount per | days after | |
| Tumor type | Cell line | dose, μg) | first dose | T/C (%) |
| Gastric | N87 | 4.00 | 27 | 19 |
| | | | 28 | 60 |
| | | | 30 | 48 |
| | | | 31 | 39 |
| | | | 33 | 39, 42* |
| | | 2.00 | 28 | 41 |
| | | | 30 | 54 |
| | | 1.00 | 28 | 55 |
| Colon | HT29 | 4.00 | 33 | 36 |
| | LOVO | 4.00 | 25 | 80 |
| | | | 29 | 47, 76* |
| Lung | L2987 | 4.00 | 35 | 1 |
| | | | 30 | 1 |
| | | 3.00 | 21 | 5 |
| | | 2.00 | 30 | 5 |
| | | 1.50 | 21 | 77 |
| | | 0.75 | 21 | 33 |
| | PC14PE6 | 4.00 | 17 | 27 |
| | | | 22 | 15 |
| | | | 29 | 14 |
| | | 2.00 | 17 | 59 |
| | | | 29 | 42 |
| | | 1.00 | 29 | 52 |
| Breast | MDAMB435/5T4 | 4.00 | 29 | 32 |
| | | | 34 | 30 |
| | | 2.00 | 29 | 45 |
| Cervical | A431 | 4.00 | 27 | 21, 35* |
| | | | 28 | 12 |
| | | 2.00 | 27 | 72 |
| | | 1.00 | 27 | 146 |
| | A431/Le^{Y} | 4.00 | 29 | <1 |
| | | 2.00 | 29 | 37 |
| | KB 8.5 | 4.00 | 22 | 27 |
| Prostate | LNCaP | 4.00 | 28 | 24 |
| | | | 29 | 7 |
| | | 2.00 | 28 | 30 |
| | | | 29 | 34 |
| | | 1.00 | 28 | 59 |
| | | 0.50 | 28 | 102 |
| | PC3MM2 | 4.00 | 29 | 33 |

### Example 5

### Accumulation of ¹²⁵I-Labeled hp67.6-AcBut-CalichDMH Conjugate

The kinetics of hp67.6-AcBut-CalichDMH in various mouse tissues and in CD33-negative A431-tumor xenografted tumor were compared. Following injection of 200 □g (20 μCi) ¹²⁵I labeled conjugate, the amount of radioactive label was measured in various tissues at 2, 6, 24, 48, 72 and 96 h (FIG. 2). The amount of radioactive material was expressed relative to the amount present in whole blood at the time of measurement (% Blood, Y1 axes in FIG. 2). Percent blood (% Blood) is given by the formula, 100×Bq per gram tissue/Bq per gram blood. In addition, the amount of radioactive material was also expressed relative to total amount of conjugate given (% ID/g, Y2 axes in FIG. 2). Only a marginal amount of 125I labeled conjugate is retained in the brain. The accumulation of conjugates in the brain did not significantly vary within 96 hours. The % Blood was on average 3.5%. Hence, this value should not be interpreted as the result of conjugate uptake by the tissue because the blood-brain barrier is impenetrable for antibodies.

During a period of 96 hours, the amount of hp67.6-AcBut-CalichDMH in tumor tissue relative to the amount in whole blood increases from 6 to 28%. This steady increase was exclusively found in tumor tissue. The % Blood-values of heart, intestine and spleen were highest at 2 hours after injection and then steadily decreased in function of time. In liver and striated muscle, the peak of the % Blood-value was at 48 h. In skin, this value reached a plateau after 24 h. The increase of the % Blood-value in tumor tissue was not solely the result of clearance of p67.6-AcBut-CalichDMH from the blood. This was evidenced by a steady increase of the % ID/g value, which is a better indicator of the absolute amount of hp67.6-AcBut-CalichDMH in the tissue.

In contrast to tumor-tissue, the % ID/g decreased in function of time in all the other tissues that were examined. Tumor tissue was thus exceptional in its capacity to retain and accumulate hp67.6-AcBut-CalichDMH. The former experiment demonstrated accumulation of the antibody in tumor tissue. The ¹²⁵I-label indicated the presence of the antibody but did not demonstrate whether the CalichDMH part of the conjugate follows a similar accumulation trend. The tissue distribution of hp67.6 conjugated to ³H-labeled CalichDMH was similar to that of ¹²⁵I labeled conjugate. Thus, the cytotoxic part of the conjugate was similarly distributed as the immunoglobulin carrier in both normal and neoplastic tissues.

### Example 6

### Passive Targeting of RITUXIMAB and G5/44 Conjugates

Calicheamicin conjugates of rituximab and g5/44 inhibit tumor growth to the same extend as hp67.6-AcBut-CalichDMH.

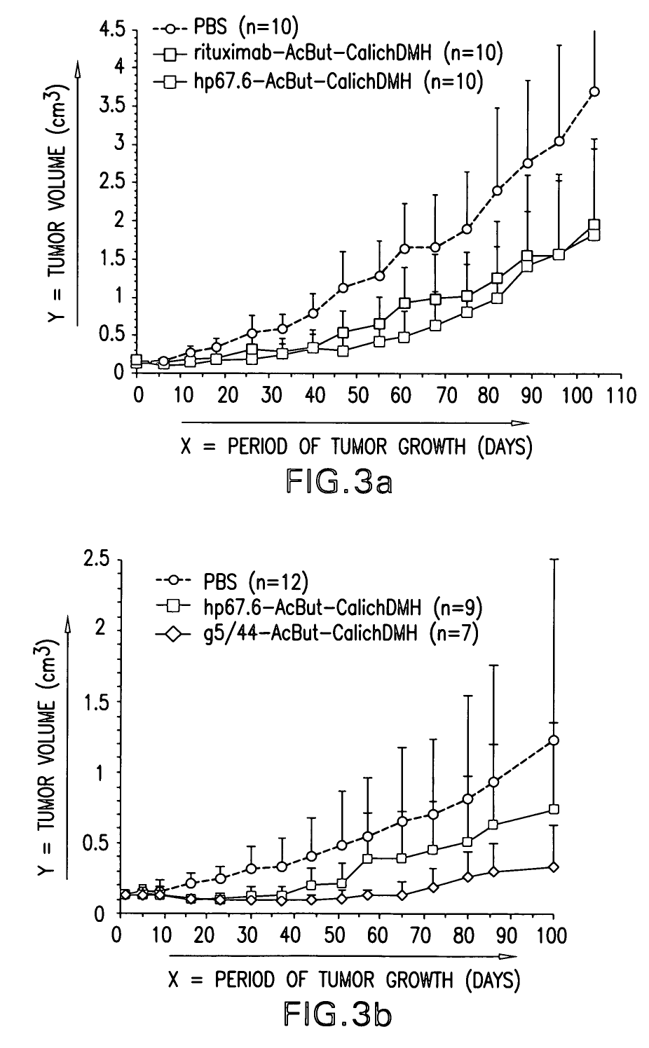

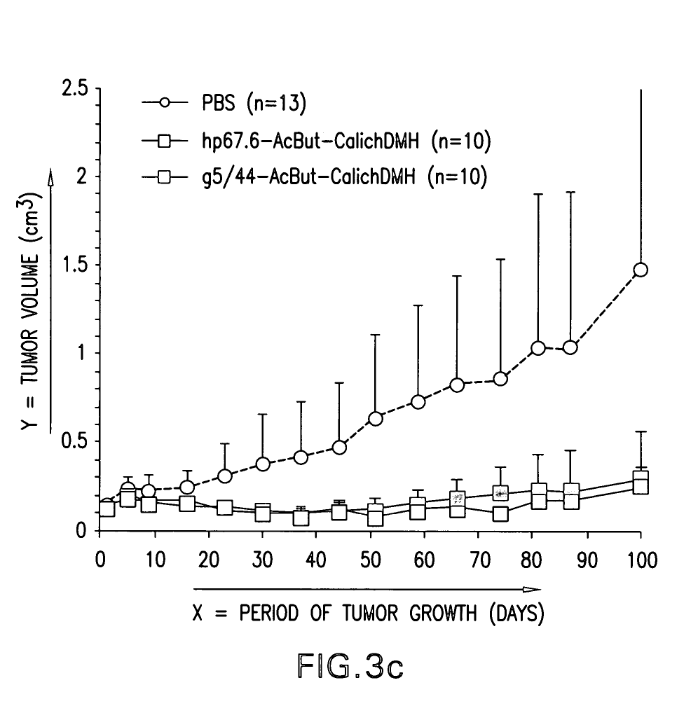

To verify whether the tumor growth inhibition caused by hp67.6-AcBut-CalichDMH was restricted to hp67.6 as a carrier for passive targeting, several experiments were conducted that compared the efficacy of hp67.6 conjugates to that of rituximab and G5/44 conjugates. None of the 3 antibodies bound with high avidity to N87 or MDAMB435/5T4. The reMCF values after probing N87 or MDAMB435/5T4 with rituximab were 0.96 and 0.89 respectively. After probing these cells with g5/44, the reMCF values were between 0.76 and 1.60. Despite the low avidity of the antibodies for the cell lines, their calichemicin conjugates caused significant inhibition of tumor growth (FIG. 3). FIG. 3 also illustrates that equivalent efficacy was achieved with the conjugates regardless of the specificity, isotype or isoelectric point of the antibody used for conjugation. Hp67.6 and g5/44 are fully humanized IgG4 molecules. Rituximab is a mouse-human IgG1 chimera. The isoelectric points of hp67.6, g5/44 and rituximab are 7.5, 8.4 and >9, respectively.

### Example 7

### Human Serum Albumin Or PEGylated Fc Conjugates

Substituting the antibody with either human serum albumin or PEGylated Fc fragments reduces the efficacy of calicheamicin conjugates.

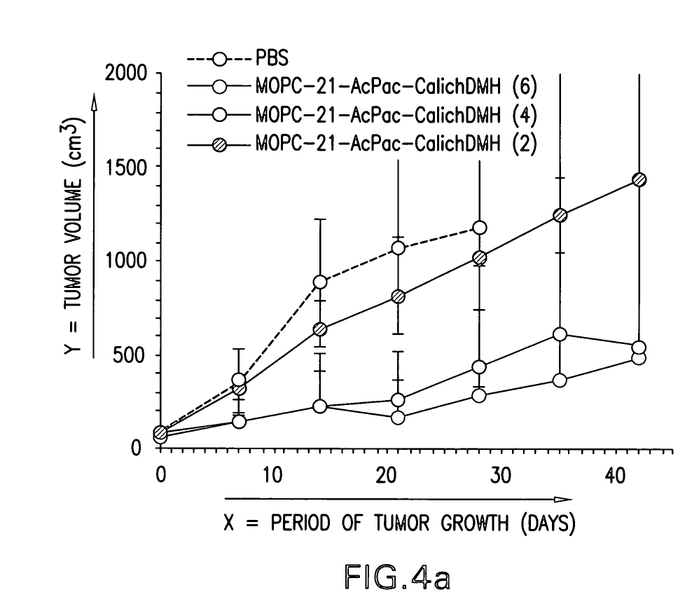

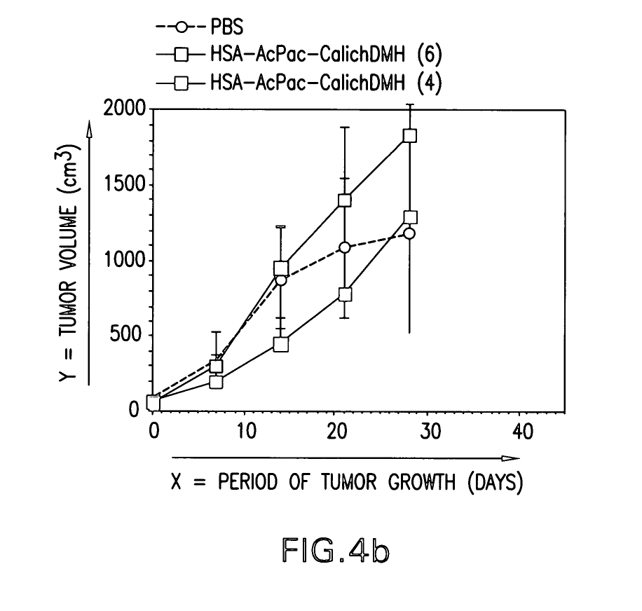

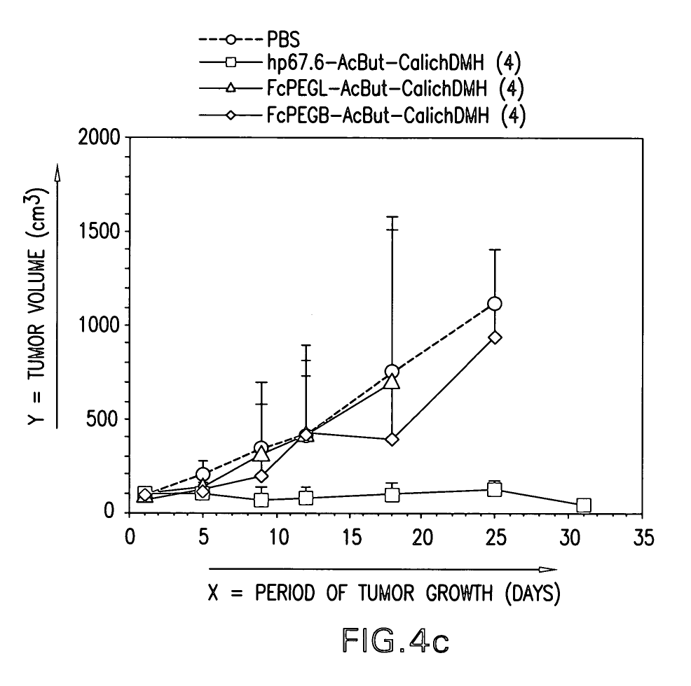

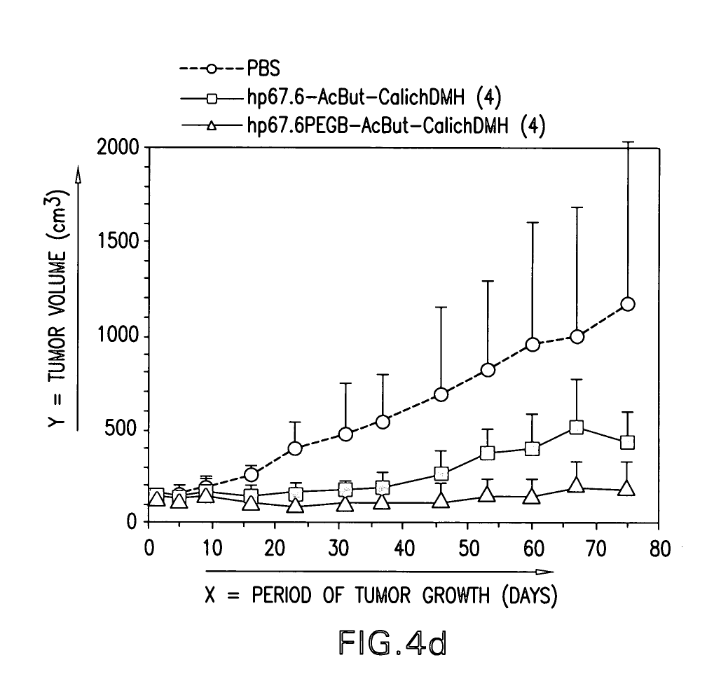

The data presented in FIG. 4 indicate that for a calicheamicin conjugate to be efficacious, neither human serum albumin nor PEGylated Fc can replace the carrier antibody. FIG. 4A shows the growth inhibition of the MOPC-21-AcPAc-CalichDMH conjugate. MOPC-21 is a mouse monoclonal antibody (IgG1) with unknown specificity that is commonly used as negative control in immunodetection methods. To conjugate calicheamicin to this mouse antibody the acid labile AcPAc linker was used. This conjugate efficacy indicates that using the AcPAc linker does not prevent oncolytic effects of the conjugate. In comparison, the efficacy of HSA-AcPAc-CalichDMH was marginal within the same experiment (FIG. 4B). Although this conjugates was more efficacious in another experiment (i.e. T/C=39% 20 days after administration of 4 μg/mouse Q4D×3), it did not have higher efficacy than the control antibody conjugate (i.e. T/C=21%). Also the fact that large interexperimental variation was observed with HSA-AcPAc-CalichDMH indicated that HSA was not as appropriate a carrier as immunoglobulin to mediate passive targeting.

The usefulness of a complete antibody was further illustrated in FIG. 4C. In this experiment, tumor growth inhibition by hp67.6-AcBut-CalichDMH is compared to the efficacy of a conjugate consisting of a PEGylated Fc fragment linked to calicheamicin by means of an AcBut linker. Two types of PEG were used to increase the Stoke's radius of the conjugate. The FcPEGL (apparent MW=300 kDa) was PEGylated with the linear N-Hydroxysuccinimidyl ester of Methoxy poly (ethylene glycol) propionic acid. FcPEGB was PEGylated with a branched form of this molecule (apparent MW=250 kDa). Regardless of the nature of the PEGylation, the Fc conjugates failed to cause any growth inhibition.

Alternatively, with a complete PEGylated (branched PEG) antibody (hp67.6PEGB, apparent MW=300 kDa) that was conjugated to calicheamicin a growth inhibition similar to that of hp67.6-AcBut-CalichDMH was observed indicating that PEGylation per se did not abrogate the efficacy of a conjugate (FIG. 4D). Taken together, the evidence presented in FIG. 4 underscores the unique propensity of whole antibody as an effective carrier of calicheamicin.

### Example 8

### Correlation With Calicheamicin Sensitivity

The degree of efficacy caused by passive targeting of p67.6-AcBut-CalichDMH correlates with the sensitivity of tumor cells to calicheamicin in vitro.

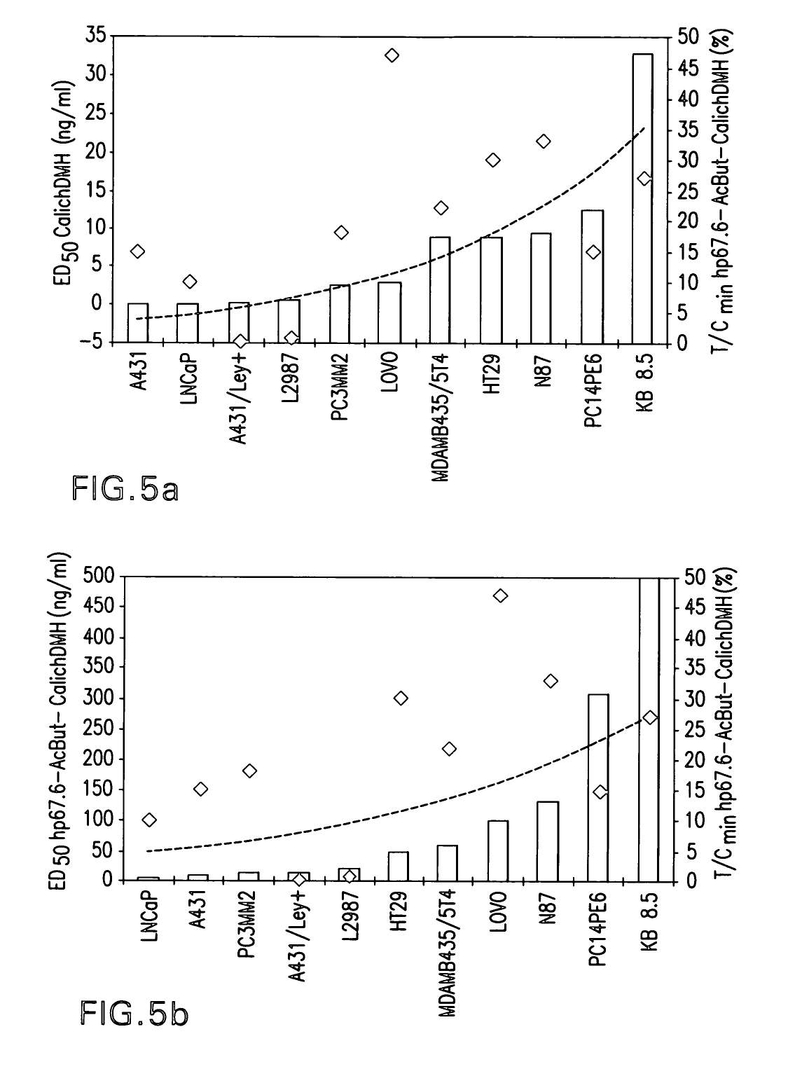

The sensitivity of 11 tumor cell lines to CalichDMH or to hp67.6-AcBut-CalichDMH was tested in vitro. The ED₅₀ of these two drugs was defined as the lowest concentration (ng/ml) that reduced the number of cells in a monolayer after 96 h to 50% of an untreated control culture. The rank order of the various cell lines was similar whether ED₅₀ of CalichDMH or ED₅₀ of hp67.6-AcBut-CalichDMH was used as a ranking criterion (compare FIG. 5A with FIG. 5B). The sensitivity of the subcutaneous xenografts to p67.6-AcBut-CalichDMH was reflected by the T/Cₘᵢₙ value. This parameter is the minimum T/C-value observed during a given experiment and reflects therefore the maximum therapeutic benefit of the conjugate determined. Hence, the T/Cₘᵢₙ value allowed a comparison of the efficacy of hp67.6-AcBut-CalichDMH on the various xenografts.

FIG. 5 demonstrates that T/Cₘᵢₙ value of these xenografts was directly proportional to the ED₅₀s determined after addition of CalichDMH (FIG. A) or hp67.6-AcBut-CalichDMH (FIG. 5B) to the reciprocal cells. This correlation suggested that sensitivity to CalichDMH was a determinant for the efficacy of hp67.6-AcBut-CalichDMH. However, the exceptionally high T/Cₘᵢₙ value found for LOVO colon carcinoma underscores that sensitivity to CalichDMH alone may be not sufficient to explain efficacy by passive targeting.

All references and patents cited above are incorporated herein by reference. Numerous modifications and variations of the present inventions are included in the above-identified specification and are obvious to one of skill in the art and are encompassed within the scope of the claims.

What is claimed is:

1. A method of treating cancer cells comprising administering to a patient in need thereof a therapeutically effective amount of a non-specific antibody conjugated to a cytotoxin, wherein the cancer cells do not express an antigen to which the non-specific antibody binds.

2. The method of claim 1, wherein the non-specific antibody is an anti-CD33 antibody and the cancer cells do not express CD33.

3. The method of claim 2, wherein the non-specific antibody is hp67.6.

4. The method of claim 1, wherein the non-specific antibody is an anti-CD22 antibody and the cancer cells do not express CD22.

5. The method of claim 4, wherein the non-specific antibody is g5/44.

6. The method of claim 1, wherein the non-specific antibody is an anti-CD20 antibody and the cancer cells do not express CD20.

7. The method of claim 6, wherein the non-specific antibody is rituximab.

8. The method of claim 1, wherein the non-specific antibody does not bind a human antigen.

9. The method of any of claims 1-8, wherein the cancer cells are gastric carcinoma, colon carcinoma, non-small cell lung carcinoma (NSCLC), breast carcinoma, epidermoid carcinoma, or prostate carcinoma cells.

10. The method of any of claims 1-9, wherein the cytotoxin is calicheamicin.

11. The method of any of claims 1-10, wherein the calicheamicin is conjugated to the non-specific antibody using a 4-(4′-acetylphenoxy)butanoic acid (AcBut) or (3-Acetylphenyl)acetic acid (AcPAc) linker.

12. The method of any of claims 1-11, wherein the antibody to the non-specific antigen conjugated to a cytotoxin is administered in combination with a bioactive agent.

13. The method of claim 12, wherein the bioactive agent is an anti-cancer agent.
